(19) United States
(12) Patent Application Publication
(10) Pub. No.: US 2003/0130248 A1
(43) Pub. Date: Jul. 10, 2003

(54) Topical anti-inflammatory compositions

(76) Inventors: Keith C. Mozzone, Limerick, PA (US); Cynthia L. Russo, Pipersville, PA (US); Linda M. Mahoney, Phoenixville, PA (US); Vincent E. Manetta, Bordentown, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/305,825

(22) Filed: Nov. 27, 2002

Related U.S. Application Data
(63) Continuation of application No. PCT/US01/17082, filed on May 25, 2001.
(60) Provisional application No. 60/208,332, filed on May 31, 2000.

(30) Foreign Application Priority Data
Oct. 14, 2000 (GB) GB 0025233.8

(51) Int. Cl. A61K 31/573

(52) U.S. Cl. 514/179

(57) ABSTRACT

An aqueous topical composition, which is essentially free of lower alcohols and oil-based components, and which contains a water-insoluble anti-inflammatory agent, such as diflorasone diacetate, and particularly to such a topical composition in the form of a gel or a lotion and its use in treating skin inflammations.

### CROSS-REFERENCE TO RELATED APPLICATIONS

This application is a continuation of PCT/US01/17082, filed May 25, 2001, which claims priority from U.S. Provisional Application No. 60/208,332, filed May 31, 2000; these applications incorporated herein by reference.

### FIELD OF THE INVENTION

This invention relates to an aqueous topical composition which contains a water-insoluble anti-inflammatory agent, for example, a corticosteroid such as diflorasone diacetate, and more particularly to such a topical composition in the form of a gel or a lotion.

Steroid compositions are useful as local or topical anti-inflammatory agents. Diflorasone diacetate is an example of a corticosteroid which is used topically for the treatment and relief of inflammatory and pruritic manifestations of corticosteroid-responsive dermatoses. Compositions of diflorasone diacetate have a topical anti-inflammatory effect on man and other mammals. For example, compositions of diflorasone diacetate are useful for the reduction of symptoms in gout arthritis, rheumatoid arthritis, rheumatoid spondylitis, osteoarthritis, psoriatic arthritis, acute superficial thrombophlebitis and painful shoulder syndromes such as peritendinitis, capsulitis, bursitis, and acute shoulder dermatitis, as well as neurodermatitis, pruritus, seborrheic dermatitis, and the like. It is particularly useful for treatment of conditions of the scalp.

### REPORTED DEVELOPMENTS

The synthesis and anti-inflammatory use of diflorasone diacetate is disclosed in U.S. Pat. Nos. 3,557,158 and 3,980,778, the disclosures of which are incorporated herein by reference. Diflorasone diacetate is identified therein as 6α,9α-difluoro-11β,17α,21-trihydroxy-16β-methyl-pregna-1,4-diene-3,20-dione 17,21-diacetate. Because diflorasone diacetate is insoluble in water, it has been difficult to formulate into aqueous compositions. The '778 patent discloses several topical compositions which, with one exception, all contain oil-based components. Such compositions may have an uncomfortable feel on the skin, and are particularly undesirable for use on the scalp. A water-based “cream-like” composition for topical use is disclosed as Example U of the '778 patent, but this example calls for dissolving the diflorasone in isopropanol which can irritate sensitive or damaged skin.

There is, therefore, a need for water-based compositions of diflorasone diacetate which are not irritating to sensitive skin.

### SUMMARY OF THE INVENTION

The present invention provides a topical therapeutic composition which comprises a therapeutically effective amount of a water-insoluble, anti-inflammatory agent dissolved in an essentially alcohol-free, water-miscible solvent for the anti-inflammatory agent, and which is essentially free of oil-based components.

A preferred group of anti-inflammatory agents for use in the composition of this invention are the corticosteroids such as, for example, diflorasone diacetate, hydrocortisone, desoximetasone, fluocinonide and betamethasone, with diflorasone diacetate being particularly preferred. Preferred embodiments of the composition of this invention are in the form of a gel or lotion which is particularly suitable for application to skin or scalp. The composition is essentially free of oil-based components which can be undesirably greasy or sticky when used on skin, particularly on the scalp or other hairy parts of the body.

The composition of the present invention can be used to treat inflammatory and pruritic manifestations of corticosteroid responsive dermatoses. The composition is non-irritating to sensitive skin and is free of solvents or other components in quantities which can cause skin irritation.

The present invention also provides a method of topically treating inflammatory and pruritic manifestations of corticosteroid responsive dermatoses by the use of the composition of the present invention.

A further aspect of the present invention is a method for formulating an aqueous topical formulation of water-insoluble treatment agents.

### DETAILED DESCRIPTION OF THE INVENTION

The present invention provides a topical therapeutic composition which comprises a therapeutically effective amount of a water-insoluble, anti-inflammatory agent dissolved in an alcohol-free, water-miscible solvent for the anti-inflammatory agent and which is essentially free of oil-based components. A preferred group of anti-inflammatory agents for use in this composition are the corticosteroids such as, for example, diflorasone diacetate, hydrocortisone, desoximetasone, fluocinonide and betamethasone, with diflorasone diacetate being particularly preferred.

The following definitions apply to terms as they are used in the present application.

A composition is considered “essentially alcohol-free” when it contains less than a nominal amount of lower alcohol (C₁ to C₄) components, including methanol, ethanol, propanol, and butanol. Although the compositions of the present invention are preferably completely free of such lower alcohol components, they may contain a nominal amount of such components, which for purposes of the present application will be considered up to about 0.01 weight percent.

“Oil-based” components are typically hydrocarbon-based and immiscible with water. A composition will be considered “essentially oil-free” when it contains less than a nominal amount of oil-based components, which for purposes of the present application will be considered less than about 1 weight percent.

A “gel” or “gel-like composition” is a single-phase, water-based and water-miscible composition which may be clear, hazy or opaque in appearance. This is distinct from an emulsion, which has an oil phase and a water phase which are combined into a single phase composition by the use of an emulsifying agent. Gels are typically thick or viscous to the point that they are usually not pourable or “free-flowing”.

A “lotion” is also a single-phase, water-based composition containing water-miscible components, which may be clear, hazy or opaque in appearance, but is typically less viscous than a gel, and is generally pourable and free-flowing.

The “skin feel” of a composition is what the patient feels during topical dosing or application of the composition.

An oily skin feel when applying an oil-based ointment, such as a petroleum jelly, is often unacceptable to a patient due to the impression that the product is sitting on the surface of the skin and will be transferred to anything, such as clothing, with which the patient comes in contact. Patients who worry about staining clothing would find such compositions unacceptable, regardless of whether the oily-feeling composition actually does transfer to the clothing. Creams are often better accepted than ointments, but they also contain an oil phase and, therefore, can sometimes leave a white film on the skin and cause an oily skin feel. In addition, cream formulations of topically-applied corticosteroids often have a lower steroid potency rating (as measured on the “Stoughton” scale) than the same steroid applied as an ointment.

On the other hand, gels and lotions primarily comprise water which generally is thickened by a hydrophilic gelling agent to form a gel matrix. As such, gels and lotions are generally water-miscible and blend well into the skin to which they are applied and do not have an oily skin feel. To the patient, they seem to be “absorbed” into the skin within a short period of time.

Because certain therapeutic agents are relatively insoluble in water they cannot be dissolved to concentrations effective for their intended therapeutic use. The anti-inflammatory agent diflorasone diacetate is such a water-insoluble therapeutic agent. In accordance with the present invention, in order to make an aqueous composition containing a therapeutically effective amount of diflorasone diacetate, the diflorasone diacetate is first dissolved in a solvent which is miscible with water. A preferred solvent for use in the present invention is propylene glycol, which is capable of dissolving the diflorasone diacetate, is non-irritating and safe to use on sensitive skin, and is miscible with water. Propylene glycol also seems to improve the skin feel of the compositions. Other suitable solvents include, but are not limited to, isopropyl myristate, isopropyl palmitate and propylene carbonate. Preferably, the composition is essentially alcohol-free, because alcohol-solvents can be irritating to sensitive skin.

The composition preferably is also essentially free of oil-based components, because such components can cause a greasy or sticky feel.

After the diflorasone diacetate is dissolved in the solvent, the solution is mixed with water to form an aqueous composition. Because these compositions are intended for medicinal use, the water is preferably purified, more preferably purified in accordance with suitable standards, such as USP grade.

The composition should comprise a solvent to dissolve the diflorasone diacetate, and water to form an aqueous composition. A preferred composition comprises from about 5 to about 80 weight percent (% w/w) solvent and from about 5 to about 80% w/w water. A more preferred composition comprises from about 20 to about 50% w/w solvent and from about 40 to about 70% w/w water. Particularly good results are obtained with compositions comprising about 40% w/w solvent and the balance water and other components.

When diflorasone diacetate is used as the anti-inflammatory agent, it is preferred that the diflorasone diacetate comprise about 0.001 to about 5% w/w, more preferably in the range of from about 0.01 to about 1% w/w. A typically preferred dosage amount of the diflorasone diacetate in the composition is about 0.05% w/w. Dosage amounts for other anti-inflammatory agents may be determined readily by one skilled in the art.

The composition may optionally contain a suitable gelling agent to obtain the desired viscosity. Suitable gelling agents include the acid form of carboxy vinyl polymers, also known as carbomers, such as those available from under the trademark Carbopol7 (The B. F. Goodrich Company). The use of such gelling agents is well known in the art. By selecting a suitable amount and molecular weight of the gelling agent the composition can be made into a gel, lotion or other desired form. Typically the composition should contain less than about 2% w/w of the gelling agent. For the present compositions, about 1% w/w Carbopol7 940, NF is particularly suitable for forming a gel composition and about 0.25% w/w Carbopol7 980 (which is similar to Carbopol 940, NF, but is characterized as “low benzene”) is suitable for forming a lotion composition.

Because the preferred gelling agent is acidic, the composition may also contain a suitable chemical base, such as sodium hydroxide, to adjust the pH to a desired level, generally about neutral or mildly acidic in a preferred pH range of about 4 to about 8.

It has been also found that a small amount of certain surfactants, particularly nonionic surfactants can improve the skin feel of the composition, giving the composition a smoother feel with better humectant (skin-soothing) properties.. Preferred nonionic surfactants include polyethylene glycol alkylphenyl ethers such as octoxynol-9, octoxynol-10, nonoxynol-9 and nonoxynol-10. In particular, nonoxynol-9, DF, which is commercially available as Igepal7 CO-630 Special (Rhodia), was found suitable for this purpose. Up to about 2% w/w nonoxynol-9 surfactant may be used for this purpose, preferably about 0.1 to 1% w/w, and a typical concentration of about 0.25% w/w.

Other additives may also be included in the compositions for their well-known purposes. A chelating agent may be added to improve the stability of the composition. A typical chelating agent is tetrasodium edetate solution, such as those commercially available under the name Dissolvine7 E-39 (AKZO Nobel), typically at concentration of from about 0.001 to about 0.01% w/w.

Another optional additive for these compositions is a defoaming agent which can be added as desired to reduce foaming. Typical defoaming agents include silicone emulsions, and may be used at the concentrations recommended by the manufacturer. he present examples use Antifoam FG-10 (Dow Coming) silicone emulsion at a concentration of from about 0.001 to about 0.1% w/w, typically about 0.015% w/w.

### EXAMPLES

The following examples are merely illustrative of the present invention and should not be considered as limiting the scope of the invention in any way. The term “USP” as used herein refers to chemicals that conform to the tolerances set forth in the United States Pharmacopoeia (U.S. Pharmacopoeial Convention, United States Pharmacopoeia and National Formulary (5^{th} ed. Supp. 1995 & Supp. 1996)). USP specifications are designed to limit contaminants that are dangerous to health. The term “NF” refers to National Formulary standards. The National Formulary is a compendium of pharmaceutical formulations widely used as a standard reference.

### Example 1—Preparation of a Topical Gel

Topical gel compositions were prepared according to the following formulation.

| Component | Weight Percent |
|---|---|
| diflorasone diacetate, USP | 0.050 |
| carboxy vinyl polymer, acid form (Carbopol7 940, NF) | 1.000 |
| propylene glycol, USP | 40.000 |
| sodium hydroxide solution, 40% w/v | 0.382 |
| tetrasodium edetate solution, DF (Dissolvine7 E-39) | 0.050 |
| silicone emulsion, DF (Antifoam FG-10) | 0.015 |
| nonoxynol-9, DF (Igepal7 CO-630 Special) | 0.250 |
| purified water, USP | balance |

The above composition was formulated at room temperature, with controls being employed to maintain the temperature at or below about 30EC throughout the manufacturing process. About 97% of the required purified water and 50% of the required propylene glycol were dispensed into a suitable stockpot or vessel. The resulting mixture was then transferred to a suitable manufacturing vessel equipped with temperature control, vacuum control, a side scraping mixer, an agitator and a rotor/stator-type shear mixer with recirculation loop. Carboxy vinyl polymer was charged into the mixing vessel with vacuum. The contents of the mixing vessel were then mixed until smooth and uniform. The tetrasodium edetate solution was then charged into the mixing vessel with vacuum. The contents of the vessel were mixed. Silicone emulsion and about 0.25% of the required purified water were then mixed in a suitable stockpot or vessel, mixed, and charged into the mixing vessel with vacuum. The contents of the mixing vessel were mixed. Nonoxynol-9 was then charged into the mixing vessel with vacuum and the contents of the vessel were mixed. Diflorasone diacetate was added to 50% of the required propylene glycol, mixed to dissolve at room temperature, and then charged into the mixing vessel with vacuum. The contents of the mixing vessel were further mixed. Sodium hydroxide solution was added to the remaining amount of the required purified water in a stockpot or vessel, mixed, and charged into the mixing vessel with a vacuum. The contents of the mixing vessel were mixed to form a gel. The gel was further mixed and a series of vacuum pulses was applied to deaerate the gel. An in-process sample was pulled for pH determination. The gel was passed through a 60 mesh screen into a suitable holding vessel. The finished bulk was then ready for filling.

### Example 2—Preparation of a Topical Lotion

Topical lotions were prepared according to the following formulation.

| Component | Weight Percent |
|---|---|
| diflorasone diacetate, USP | 0.050 |
| carboxy vinyl polymer, acid form (Carbopol7 980) | 0.250 |
| propylene glycol, USP | 40.000 |
| sodium hydroxide solution, 40% w/v | 0.035 |
| tetrasodium edetate solution, DF (Dissolvine7 E-39) | 0.050 |
| silicone emulsion, DF (Antifoam FG-10) | 0.015 |
| nonoxynol-9, DF (Igepal7 CO-630 Special) | 0.250 |
| purified water, USP | balance |

As in Example 1, the temperature was maintained at or below about 30 EC throughout the manufacturing process. About 97% of the required purified water and 50% of the required propylene glycol were dispensed into a suitable stockpot or vessel. The resulting mixture was then transferred to a suitable manufacturing vessel equipped with temperature control, vacuum control, a side scraping mixer, an agitator and a rotor/stator-type shear mixer with recirculation loop. Carboxy vinyl polymer was charged into the mixing vessel with vacuum. The contents of the mixing vessel were then mixed until smooth and uniform. The tetrasodium edetate solution was then charged into the mixing vessel with vacuum. The contents of the vessel were mixed. Silicone emulsion and about 0.25% of the required purified water were then mixed in a suitable stockpot or vessel, mixed, and charged into the mixing vessel with vacuum. The contents of the mixing vessel were mixed. Nonoxynol-9 was then charged into the mixing vessel with vacuum and the contents of the vessel were mixed. Diflorasone diacetate was added to 50% of the required propylene glycol, mixed to dissolve, and then charged into the mixing vessel with vacuum. The contents of the mixing vessel were further mixed. Sodium hydroxide solution was added to the remaining amount of the required purified water in a stockpot or vessel, mixed, and charged into the mixing vessel with a vacuum. The contents of the mixing vessel were mixed to form a lotion. The lotion was further mixed and a series of vacuum pulses was applied to deaerate the lotion. An in-process sample was pulled for pH determination. The finished bulk was then ready for filling.

### Comparative Example

For a comparison of physical properties, a test sample was prepared in accordance with the formulation set forth in U.S. Pat. No. 3,980,778 as Example U, except that the active agent diflorasone diacetate, which would have no significant effect on the physical properties, was not included.

| Component | Amount | |
|---|---|---|
| liquid dimethylpolysiloxane | 25.0 | ml |
| isopropanol | 22.0 | ml |
| carboxypolymethylene polymer (Carbopol7 940) | 0.75 | g |
| di-isopropanolamine | 0.75 | ml |
| distilled water qsad | 100.0 | g |

In accordance with the preparation method set forth in the '778 patent, the isopropanol was combined with the water, the carboxymethylene polymer was added and the mixture stirred until uniform. The '778 patent indicates that the di-isopropanolamine should be added and the mixture stirred until a smooth gel is obtained. While stirring, the liquid dimethylpolysiloxane should be added slowly and the composition mixed until uniform. The patent states that this will form a “cream-like material” which “is suitable for topical anti-inflammatory applications”.

Upon addition of the last ingredient, liquid dimethylpolysiloxane, the formulation went from a relatively smooth opaque gel to a soupy, lumpy, opaque inconsistent mass. It was not a “cream-like material”, as indicated in the '778 patent. This was not surprising, because the liquid dimethylpolysiloxane, which comprises about 25% by volume of the composition, is hydrophobic and water-immiscible. It would not be expected that an otherwise aqueous-based, hydrophilic gel could accommodate 25% of a hydrophobic substance without an emulsifier. The non-homogeneous composition had an oily feel on the skin and a profoundly distinctive odor of isopropyl alcohol, both of which would be unacceptable for purposes of the present invention.

Furthermore, when this comparative example was allowed to sit at room temperature for several days, the formulation separated into distinct phases, with the liquid dimethylpolysiloxane sitting in liquid form on the top. Without an emulsifier, this formulation was found to be physically unstable.

What is claimed is:

1. A topical anti-inflammatory therapeutic composition comprising a therapeutically effective amount of diflorasone diacetate dissolved in an essentially lower alcohol-free, water-miscible solvent for the diflorasone diacetate, wherein said composition is essentially free of oil-based components.

2. The composition of claim 1 wherein said composition comprises an amount of diflorasone diacetate in a range from about 0.001 to about 5% w/w.

3. The composition of claim 2 wherein said amount of diflorasone diacetate is about 0.05% w/w.

4. The composition of claim 1 wherein said solvent is selected from the group consisting of propylene glycol, isopropyl myristate, isopropyl palmitate, propylene carbonate and combinations thereof.

5. The composition of claim 4 wherein said solvent is propylene glycol.

6. The composition of claim 1 comprising about 5 to about 80% w/w solvent.

7. The composition of claim 6 comprising about 5 to about 80% w/w propylene glycol.

8. The composition of claim 7 comprising about 40% w/w propylene glycol.

9. The composition of claim 1 comprising a gelling agent.

10. The composition of claim 9 wherein said gelling agent is a carboxy-vinyl polymer.

11. The composition of claim 1 in the form of a gel or a lotion.

12. The composition of claim 1 further comprising a surfactant in an amount effective to improve the skin feel of the composition.

13. The composition of claim 12 wherein said surfactant is nonoxynol-9.

14. The composition of claim 1 further comprising one or more components selected from the group consisting of pH adjusting agents, chelating agents and defoaming agents.

15. A gel-like composition in accordance with claim 1 comprising:

| Component | Weight Percent |
|---|---|
| diflorasone diacetate, USP | 0.05 |
| Carbopol 940 ® carboxy vinyl polymer, acid form, NF | 1. |
| propylene glycol, USP | 40. |
| sodium hydroxide solution, 40% w/v | 0.382 |
| tetrasodium edetate solution, DF | 0.05 |
| antifoam silicone emulsion, DF | 0.015 |
| nonoxynol-9, DF | 0.25 |
| and purified water, USP | balance. |

16. A lotion composition in accordance with the claim 1 comprising:

| Component | Weight Percent |
|---|---|
| diflorasone diacetate, USP | 0.05 |
| Carbopol 980 ® carboxy vinyl polymer, acid form, NF | 0.25 |
| propylene glycol, USP | 40. |
| sodium hydroxide solution, 40% w/v | 0.035 |
| tetrasodium edetate solution, DF | 0.05 |
| antifoam silicone emulsion, DF | 0.015 |
| nonoxynol-9, DF | 0.25 |
| and purified water, USP | balance. |

17. A method of treating inflammatory manifestations on the skin of a patient comprising topically administering an anti-inflammatory effective amount of the composition of claim 1 to said patient.

18. The method of claim 17 wherein said inflammatory manifestation is a corticosteroid-responsive dermatosis.

19. The method of claim 17 wherein said skin of the patient is hairy.
